(19) United States
(12) Patent Application Publication
(10) Pub. No.: US 2021/0393367 A1
(43) Pub. Date: Dec. 23, 2021

(54) Immobilization System Having Bite-Block Stabilization and Method of Using Same

(71) Applicants: Matthew Calhoun, Heath, OH (US); Mary Ellen Devault, Newark, OH (US)

(72) Inventors: Matthew Calhoun, Heath, OH (US); Mary Ellen Devault, Newark, OH (US)

(21) Appl. No.: 17/391,263

(22) Filed: Aug. 2, 2021

Related U.S. Application Data
(63) Continuation-in-part of application No. 15/989,379, filed on May 25, 2018, now abandoned.

(51) Int. Cl. A61B 90/16; A61N 5/10

(52) U.S. Cl. CPC A61B 90/16; A61N 2005/1097; A61N 5/10

(57) ABSTRACT

An immobilization system, especially for use on the human head and neck area, is described in various embodiments. The system may include a bite block immobilization system having a bite block with a dental interface releasably engageable with a plurality of teeth of a patient, and a retention mask. The dental interface releasably engages the teeth of a patient and allows very highly accurate repositioning during multiple procedures. In some embodiments, the bite block may have an integral bite block airway passage, allowing a patient to have unobstructed breathing both during molding of the retention mask and during subsequent use of the system, and may include a tongue diverter, that may, by way of example only and not limitation, be used to divert the tongue away from a therapeutic radiation beam. Steps for utilizing the system are further described.

### CROSS-REFERENCE TO RELATED APPLICATIONS

The continuation-in-part application claims the benefit of U.S. patent application Ser. No. 15/989,379, filed May 25, 2018.

### STATEMENT REGARDING FEDERALLY SPONSORED RESEARCH OR DEVELOPMENT

Not applicable.

### TECHNICAL FIELD

The present disclosure relates generally to an immobilization system, in particular, for making an anatomically high-accuracy moldable immobilization system having a bite-block stabilization feature, well-suited for patient immobilization during radiotherapy or other applications requiring a high degree of accuracy in patient positioning. This is of particular importance for radiotherapy of the head and neck region.

### BACKGROUND OF THE INVENTION

The present invention relates to an immobilization system, for persons whose bodies or body parts are required to be retained in a particular position or attitude. In particular, a need exists for a simple and easy to use immobilization system that allows repetitive patient positioning with a high degree of accurate repeatability across time, for example, wherein radiation treatments must be directed at a certain anatomical point across different times. Such precise positioning of the head and neck area, as might be required for radiotherapy of that region, is particularly problematic due to difficulty in immobilizing the area, while maintaining the patient's ability to breath, and with a reasonable degree of patient comfort.

The use of low-temperature thermoplastics for patient positioning is well-known and dates back to splinting devices invented in the 1960's (Larson, U.S. Pat. No. 5,540,876). Splints are heated, usually in hot water, to a temperature of about 160° F., whereupon they become pliable and can be molded by hand directly on the patient's body part. These devices are well known in the field of occupational therapy and include splints with padding or cushioning material laminated to the thermoplastic to provide comfort against the patient's skin.

Plastic materials have been successfully used in the past for making splints, casts and the like. U.S. Pat. No. 3,490,444 describes the use of thermoplastic polydienes like transpolyisoprene and transpolychloroprene, which melt between 140° F. (60° Celsius) and 212° F. (100° Celsius), and which harden by crystallization at about 140° F. (60° Celsius), such that these plastics can be formed for use as a body supporting member. Poly (epsilon-caprolactone) (PCL) has also been found to be an excellent splint or cast material (U.S. Pat. No. 4,144,223). Polyurethanes based on prepolymers of poly (epsilon-caprolactone) have also been used (U.S. Pat. No. 4,316,457).

As described in earlier patents, the polymers can be heated in hot water at a temperature usually exceeding 122° F. (50° Celsius) and up to about 212° F. (100° Celsius), whereby they become soft, self-adherent and sufficiently pliable to be deformed and shaped as a cast, splint or protective device. When allowed to cool in air to about 140° F. (40° Celsius), the materials will remain pliable, moldable and cohesive for a period of several minutes, exhibiting a hysteresis, as described in U.S. Pat. No. 3,490,444. During this time the splint, cast or device can be molded directly to the patient without discomfort, and the shaped plastic sets hard by crystallization to assume a rigid form as a useful body support member or protective device.

In the field of radiation therapy, precise patient positioning is essential for treatment accuracy. An additional requirement is that patients be precisely re-positioned for repeated radiation treatments. Repositioning to an accuracy within millimeters, or even less, is generally desirable. This requires the positioning to be reproduced accurately each time the patient undergoes a treatment. Low-temperature thermoplastic masks in conjunction with other positioning methods are often used for such positioning. Masks are heated to a temperature of about 160° F. (71° C.) and formed directly on to the patient's head or other body part. The masks may be affixed to a table supporting the patient and cooled to form a firm mask holding the patient steady for treatment. After treatment, the mask may be removed. When the patient returns for the next treatment, the mask is releasably reattached, holding the patient in a reproduced position for treatment.

Various masks are used for radiation therapy treatments, including stereotactic head masks holding the top and bottom of the patient's head (Vilsmeier, U.S. Pat. No. 5,702,406). In the field of moldable head and neck masks, a particular problem is holding the mask, when heated, to conformance around the patient's facial and head area for the short period of time required for the mask to cool and assume a stable shape. A tension exists between holding the mask accurately to conform to these features, while maintaining a reasonable degree of patient comfort.

Another goal of patient immobilization systems for radiation therapies is the avoidance of as much mass surrounding the patient's body as possible, as the mass tends to attenuate a radiotherapy beam. Therefore, the avoidance of unnecessary bulk in the immobilization system confers an advantage over more bulky systems. However, at the same time, systems have found it difficult to achieve adequate stabilization without using relatively high-mass immobilization devices.

### SUMMARY OF THE INVENTION

The disclosed invention relates to methods to for making an immobilization system. Especially for those systems intended for use on the human head and neck area, there may be a retention mask, molded to the face or head contours of the patient, holding the desired body parts firmly to the formable support. This allows for high reproducibility in positioning.

The system may include a bite block immobilization system having a bite block with a dental interface releasably engageable with a plurality of teeth of a patient. The dental interface may be supplied with the system or may be added at the time of use by a user. The dental interface releasably engages the teeth of a patient and allows very highly accurate repositioning during multiple procedures. In some embodiments, the bite block may have an integral bite block airway passage, allowing a patient to have unobstructed breathing both during molding of the retention mask and during subsequent use of the system.

In yet another series of embodiments, the bite block releasably cooperates at a tongue diverter receiver with a tongue diverter, that may, by way of example only and not limitation, be used to divert the tongue away from a therapeutic radiation beam. Steps for employing the system are further described below.

### BRIEF DESCRIPTION OF THE ILLUSTRATIONS

Without limiting the scope of the as disclosed herein and referring now to the drawings and figures:

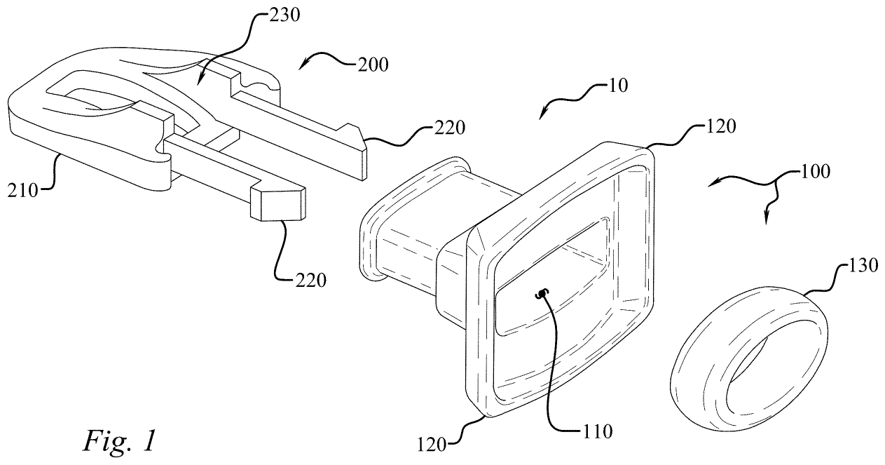

FIG. 1 is an unassembled elevated perspective view of an embodiment of the current specification;

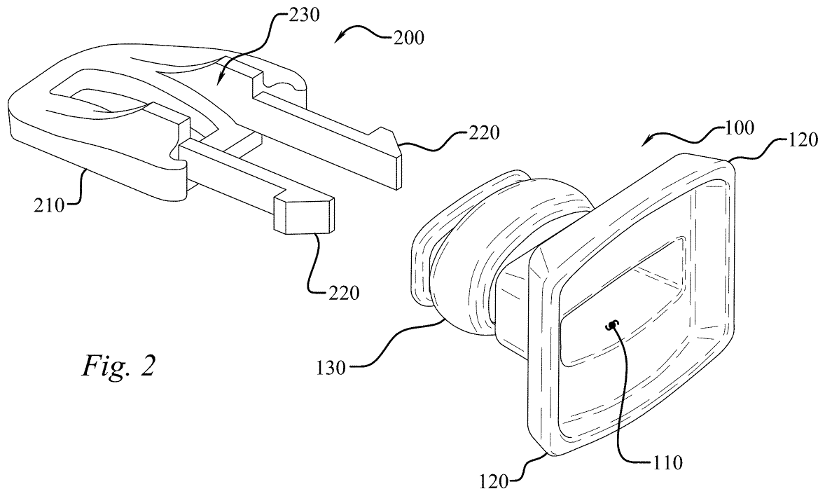

FIG. 2 is an unassembled elevated perspective view of a detail of a portion of the embodiment of FIG. 1;

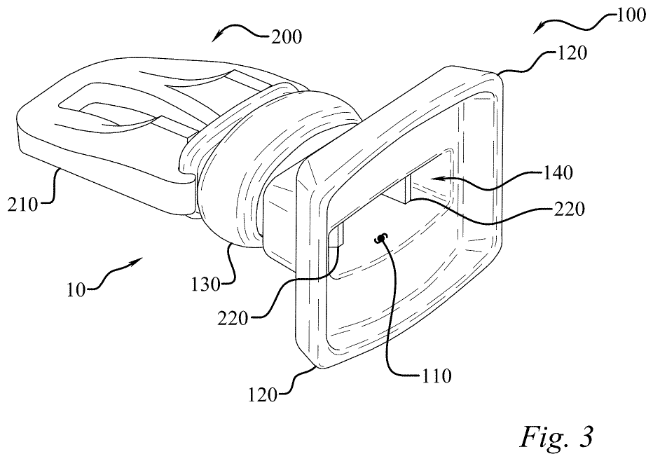

FIG. 3 is an assembled elevated perspective view of a further detail of a portion of the embodiment of FIG. 1;

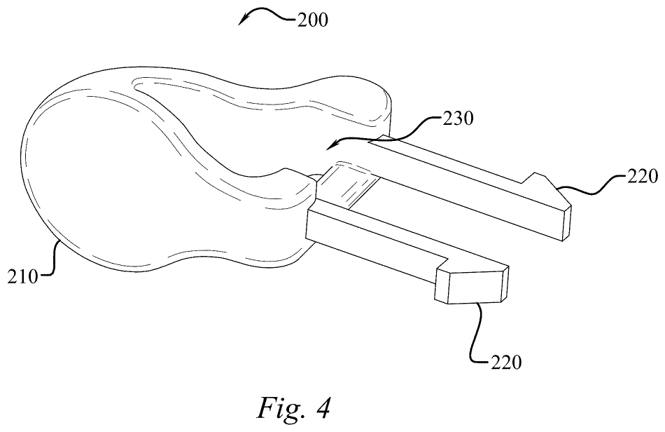

FIG. 4 is a further elevated perspective view of a detail of a portion of the embodiment of FIG. 1;

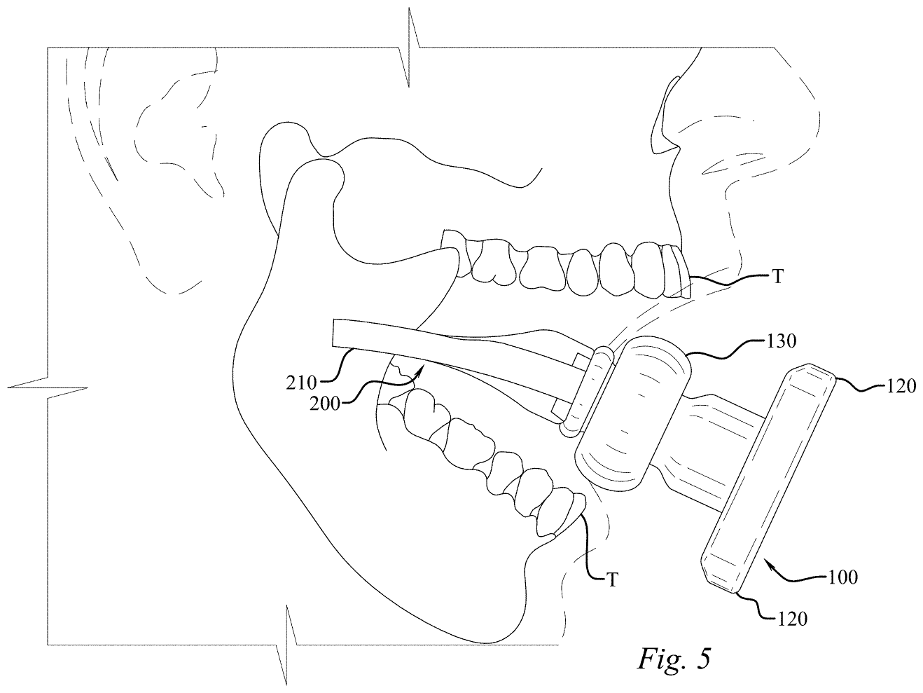

FIG. 5 is a lateral elevation view of an assembled embodiment of the device of FIG. 1 seen being placed in operating position between a patient's mandible and maxilla;

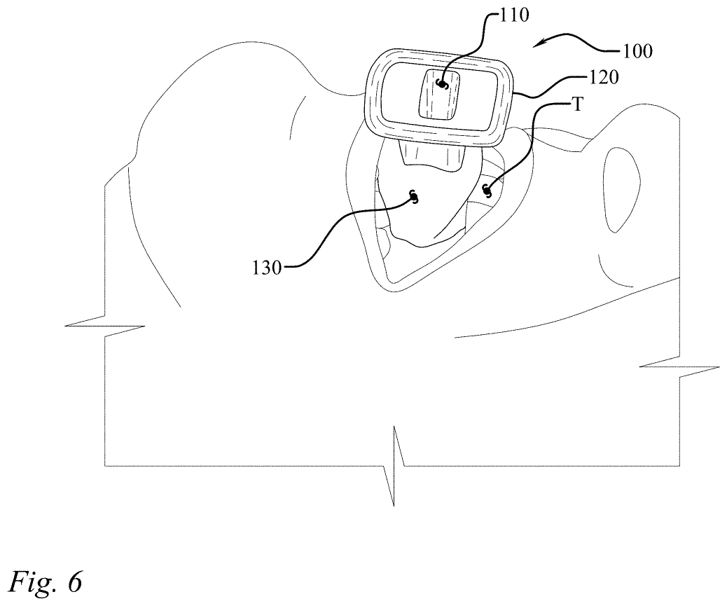

FIG. 6 is an elevated perspective view of an embodiment of the device of FIG. 1 in place in an operating position between a patient's mandible and maxilla;

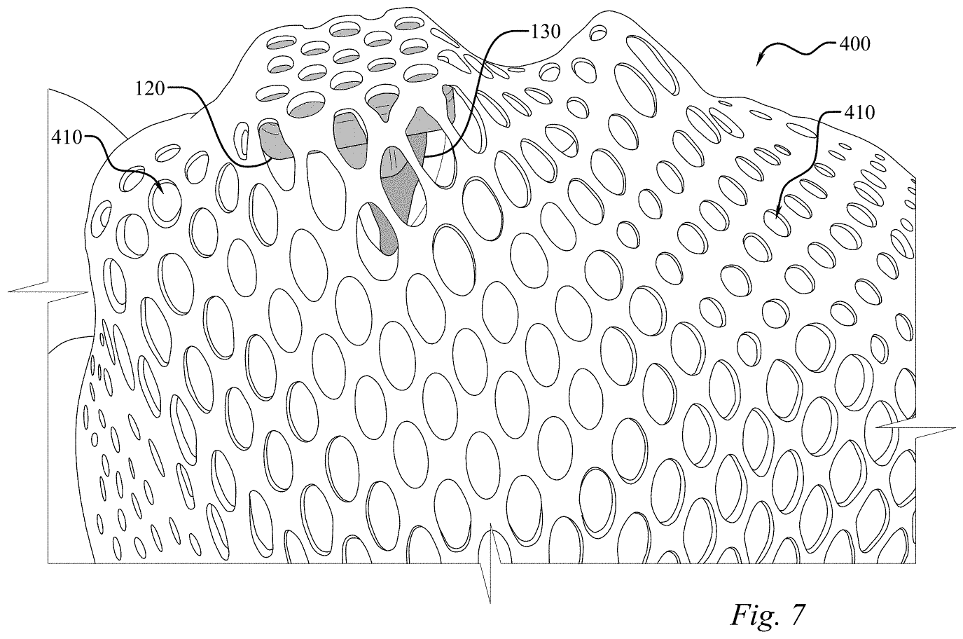

FIG. 7 is an elevated perspective view of a prior-art facial positioning mask placed over the embodiment of the device of FIG. 1 in an operating position;

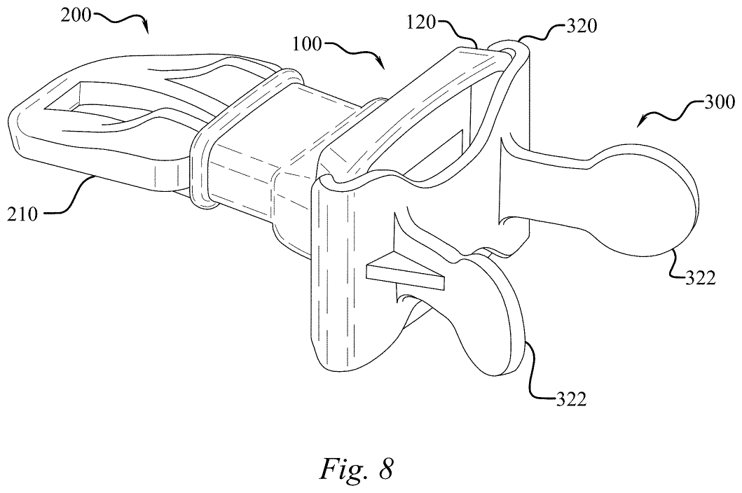

FIG. 8 is a further embodiment of the device of FIG. 1, further showing a digital manipulator in place in a non-operating position;

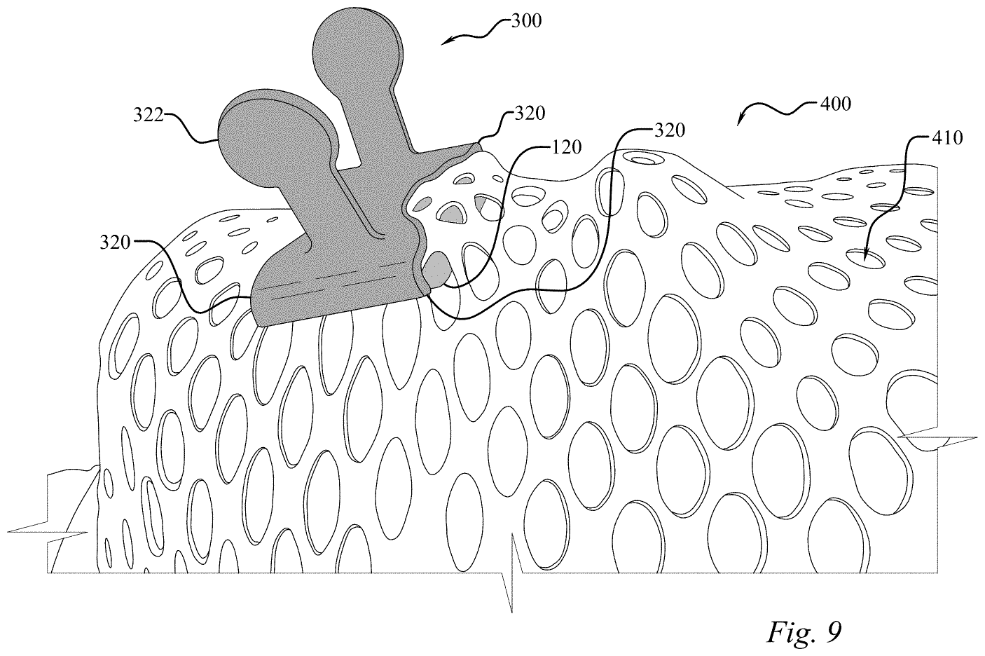

FIG. 9 is a further embodiment of the device of FIG. 1, further showing a digital manipulator in place in an operating position; and

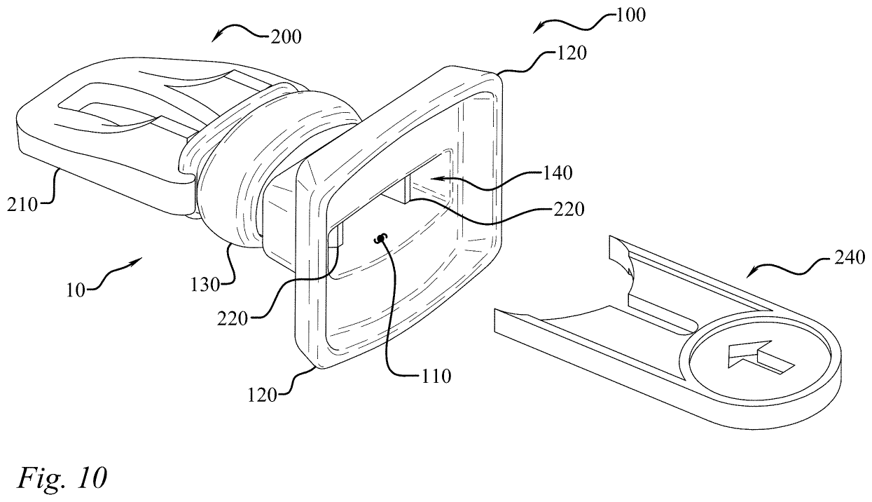

FIG. 10 is a further embodiment of the device of FIG. 1, showing a release tool in a disengaged state relative to a bite block and tongue diverter; and

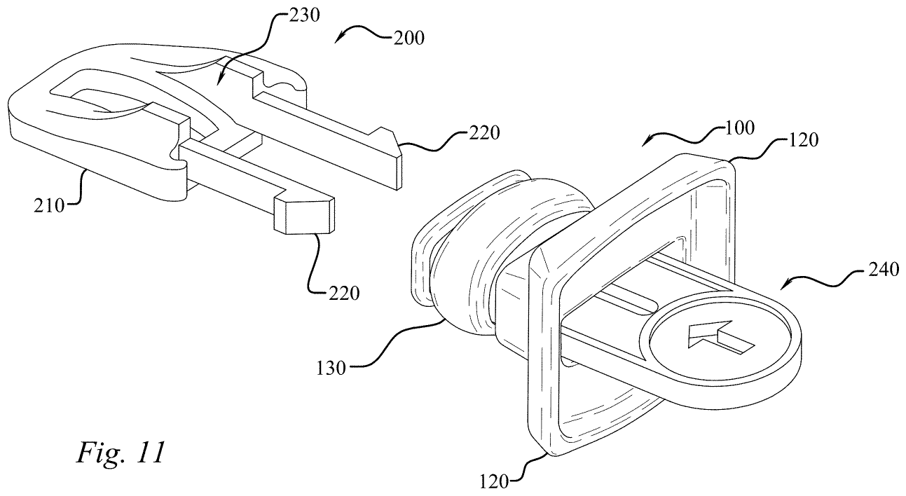

FIG. 11 is a further embodiment of the device of FIG. 1, showing a release tool in an engaged state relative to a bite block, having released a tongue diverter.

These illustrations are provided to assist in the understanding of the exemplary embodiments of the method of forming an immobilization system having a bite block stabilization feature and materials related thereto described in more detail below and should not be construed as unduly limiting the specification. In particular, the relative spacing, positioning, sizing and dimensions of the various elements illustrated in the drawings may not be drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

As seen in FIGS. 1-11, an immobilization system (10) for highly reproducible patient positioning is presenting in various embodiments, all of which would be readily understood by one skilled in the art. In one embodiment, seen well in FIGS. 1, 2, 7 and 9, the system (10) may include a bite block immobilization system (10) having a bite block (100) having a dental interface (130) releasably engageable with a plurality of teeth of a patient, and a bite block-retention mask interface (120) releasably engageable with a retention mask (400). The dental interface (130) may include a moldable dental impression putty in some embodiments, while in others, may include a moldable thermoplastic ring. The dental interface may be supplied with the system (10) or may be added at the time of use by a user. The dental interface (130) releasably engages the teeth of a patient and allows very highly accurate repositioning during multiple procedures.

The retention mask (400), seen well in FIG. 9, may be releasably and memorably moldable to the contours of a face of the patient. Such an embodiment, seen well in FIGS. 8 and 9, may also have a retainer means (300) cooperating with the bite block (100) and the retention mask (400) to releasably secure the bite block (100) and the retention mask (400). As would be known to one skilled in the art, the retention mask (400) may be secured to a backing board by any feasible releasable securing means for greater stability and reproducibility.

In some embodiments, seen well in FIGS. 7 and 9, the retention mask (400) may have a plurality of mask fenestrations (410). In other embodiments, seen well in FIGS. 1-3, the bite block (100) may have an integral bite block airway passage (110). This allows a patient to have unobstructed breathing both during molding of the retention mask (400) and during subsequent use of the system (10).

In yet another series of embodiments, the bite block (100), seen well in FIGS. 1-4 and 8, releasably cooperates at a tongue diverter receiver (140) with a tongue diverter (200), while in others, seen well in FIGS. 2-3, the tongue diverter receiver (140) may be releasably engageable with a tongue diverter retainer (220) to releasably engage the tongue diverter (200) and the bite block (100).

The tongue diverter (200) may further include as seen well in FIG. 4, a diverter airway (230) and may also include, a seen well in FIGS. 3-5, a lingual interface (210). The lingual interface (200), as would be known by one skilled in the art, may divert the tongue of a patient in a lateral, cephalic, or caudal direction, or in some combination of multiple directions.

In various embodiments, seen by way of example and not limitation only in FIGS. 8-9, the retainer means (300) may be releasably engageable with bite block (100) and the retention mask (400) by means of an interface retainer (320) releasable securing the bite block (100) to the retention mask (400). Such an interface retainer (320) may be a humanly releasable digital manipulator (322). In still other embodiments, seen well in FIGS. 10-11, a tongue diverter removal tool (240) may be releasably engageable with the tongue diverter receiver (140) to releasably disengage the tongue diverter (200) and the bite block (100).

In another series of embodiments, a bite block immobilization system (10) may include a bite block (100) having an integral bite block airway passage (110), and a dental interface (130) releasably engageable with a plurality of teeth of a patient. The bite block (100) may be releasably cooperating at a tongue diverter receiver (140) with a tongue diverter (200) having a lingual interface (210) and a diverter airway (230). Such a tongue diverter receiver (140) may be releasably engageable with a tongue diverter retainer (220) to releasably engage the tongue diverter (200) and the bite block (100). Such embodiments may also include a bite block-retention mask interface (120) releasably engageable with a retention mask (400), and such a retention mask (400) may be being releasably and memorably moldable to the contours of a face of the patient. Further, there may be a retainer means (300) cooperating with the bite block (100) and the retention mask (400) to releasably secure the bite block (100) and the retention mask (400).

In some embodiments, the tongue diverter (200) may further include a lingual interface (210) that may divert the tongue of a patient in a lateral, cephalic, or caudal direction, or allow a diversion in multiple axes of direction. In other embodiments, the retainer means (300) may be releasably engageable with a bite block (100) and the retention mask (400) by means of an interface retainer (320) releasable securing the bite block (100) to the retention mask (400), and the interface retainer (320) may be a humanly releasable digital manipulator (322). This obviates the need for a user to maintain pressure over the retention mask (400) while the retention mask (400) is converted from a more flexible, to a less flexible, state, most commonly by allowing the retention mask (400) to thermally cool.

One skilled in the art would recognize the most optimal methods of using the immobilization system. Steps for use might include; placing a bite block (100) between the maxilla and mandible of a human patient, and then placing a thermally moldable retention mask (400), in a moldable state, over at least a portion of the face of the human patient. Next, a user might secure the retention mask (400) to the bite-block (100) with a releasably engageable retainer means (300). Lastly, a user might observe or facilitate the conversion of the retention mask (400) to a fixed state, which could occur by any known method of converting the retention mask (400) from a flexible to a more rigid state, such as thermal hardening or chemical curing, and then, disengaging the releasably engageable retainer means (300).

Securing the releasable engageable retainer means (300) to the bite-block could be accomplished by the application of digital human pressure, and a subsequent step of may include the step of disengaging the releasable engageable retainer means (300) to the bite-block by the application of digital human pressure.

In a further series of embodiments, and seen well in FIGS. 1-11, what is claimed is a bite block immobilization system (10) that may include, a bite block (100) having an integral bite-block airway passage (110) and a dental interface (130), substantially ring-shaped and oriented parallel to a direction of bite and having a maxillary and a mandibular dental engagement, releasably and directly engageable with a plurality of the maxillary and mandibular teeth of a patient, and a bite-block retention mask interface (120), all seen well in FIGS. 1 and 2. The bite block (100) is releasably engageable with a retention mask (400), seen well in FIGS. 7 and 9.

The retention mask (400), known well in the art, may be thermoformable and may have at least two generally parallel opposing surfaces, a first surface in contact with the skin of the patient during use, and a second surface in contact with an ambient atmosphere during use, again seen well in FIGS. 7 and 9.

The bite block (100) contacts the retention mask (400) on the first surface of the retention mask (400), as seen in FIG. 7, without extending partially to, to, or through the second surface of the retention mask (400).

A retainer means (300) cooperates with the bite block (100) and the second surface of the retention mask (400) to releasably secure the bite block (100) and the retention mask (400), as seen in FIG. 9, and the retainer means (300) contacts the retention mask (400) on the second surface of the retention mask (400), and does not extend partially to, to, or through to the first surface of the retention mask (400).

In some embodiments, the dental interface (130) is integrally formed with the bite-block as an outside surface of the bite block (100), as seen in FIG. 1. In others, the dental interface (130) is moldable and fully removable from the bite block (100). In practice, such an interface may be formed of common dental impression putty, and may be thermally or chemically curable from a malleable to a more formed state

In some embodiments the dental interface (130) may contact eight or fewer teeth as found in a normal human full dentition set, while in others, wherein the dental interface (130) contacts 12 or fewer teeth as found in a normal human full dentition set, as can be visualized by FIG. 5. This is an important advance over prior art devices, which frequently use tray-type dental interfaces. Such trays increase the bulk of material that must be placed in a patient's mouth and tend to attenuate or scatter the beam the beam of high intensity radiation devices that are frequently used in conjunction with such immobilization systems. The additional intra-oral bulk of a tray-based system also can increase patient discomfort. The present system (10) in its various embodiments, minimizes such bulk and yet provides sound anchoring of the system (10).

The device may have a tongue diverter receiver (140) on the bite block (100) that is releasably engageable with a tongue diverter retainer (220) on a tongue diverter (200) to releasably engage the tongue diverter (200) and the bite block (100). Additionally, the tongue diverter (200) may exert a direct force on the tongue when the tongue diverter retainer (220) is in place in the bite block (100) during use, in a direction selected from the group of directions consisting of: to a patient's left relative to an imaginary vertical passing through the bite block (100), to a patient's right relative to the imaginary vertical passing through the bite block (100), caudally, or a combination of these, as seen in FIGS. 3, 4, 5, 8, 10 and 11.

This is yet another advance, in various embodiments, by the present design(s). Moving the tongue out of the treatment path can reduce the radiation dose received by the tongue, which can lower morbidity effects such as loss of tase and soreness of the tongue surface, all co-morbidities that are well known to those skilled in the art. Such tongue diversion may be particularly important during “flash therapy,” in which radiation of higher dosage is administered at less frequent intervals, and a carefully targeted beam path is particularly important.

To facilitate such gentle movement of the tongue, the tongue diverter (200) may have a lingual surface shape selected from the group of lingual surface shapes consisting of; curved to the patient's left relative to an imaginary vertical passing through the bite block (100), curved to the patient's right relative to the imaginary vertical passing through the bite block (100), and substantially orthogonal to the imaginary vertical passing through the bite block (100). This latter configuration is seen well in FIG. 5, where the tongue diverter (200) while generally orthogonal to the imaginary vertical passing through the bite block (100), has a slightly curved shape to better match the anatomy of the human tongue.

In yet other embodiments, seen well in FIG. 4, the tongue diverter (200) may have at least two lingual surface shapes selected from the group of lingual surface shapes consisting of; curved to the left relative to the imaginary vertical passing through the bite block (100), curved to the right relative to the imaginary vertical passing through the bite block (100), and substantially orthogonal to the imaginary vertical passing through the bite block (100).

Seen well in FIG. 11, there may be, in some embodiments, a tongue diverter removal tool (240) that is releasably engageable with the tongue diverter receiver (140) to releasably disengage the tongue diverter (200) and the bite block (100) while the bite block (100) remains engaged with the maxillary or mandibular teeth, or both the maxillary and mandibular teeth. This feature will be discussed after the following disclosures relative to such embodiments.

As will be described more fully below, the fully configured system (10) allows the bite block (100) and the retention mask (400) to be easily separated, even after the retention mask (400) may be fully formed. The retainer means (300) may be a manually releasable resilient clip. The retainer means (300) may be releasably engageable with the bite block (100) and the retention means (400) by means of an interface retainer (320) releasably securing the bite block (100) to the first surface of the retention mask (400) by pinching the retention mask (400) against the bite block (100).

In the embodiments under discussion, the retainer means (300) may mold the first and second surfaces of the retention mask around a bite block-retention mask interface lateral lip (120) formed on a lateral and anterior aspect of the bite block (100), as seen well in FIGS. 7, 8 and 9. One skilled in the art will envision, from this description and these figures, that bite block (100) is disengageable, after the bite block (100) and the retention mask (400) are assembled into a usable conformation, from the retention mask (400), without residual effect on the retention mask (400) structure, and that therefore the retainer means (300) may be fully releasable from the system (10) during therapeutic medical interventions.

As one skilled in the art would be able to visualize, with particular reference to FIGS. 8 and 9, the bite block (100) may reversibly snap into a pocket formed in the first surface by molding of the retention mask (400) around the bite block (100) by the retainer means (300). Because the retention mask has some degree of resiliency, a relatively rigid bite block (100) can slightly deform the pocket it has formed in the retention mask (400) as it is snapped in and out of the mask, without damaging the retention mask (400). Because no puncture or holes need be made in the retention mask (400), the structural integrity of the retention mask (400) is maintained at all times.

Returning now to the ability, in some embodiments, of the system allowing the tongue diverter (200) to be released from the bite block (100) while the bite block remains engaged with the teeth, one skilled in the art can envision the following scenario. Particularly, but not necessarily, if the patient has a painful mouth condition, it may be uncomfortable for the patient if the entire system (10) is removed from the patient in a single motion. If desired, the retention mask (400) may be snapped off the bite block (100) while the bite block (100) is still engaged with the teeth, and the retention mask (400) set aside. The tongue diverter removal tool (240) may then be engaged with the tongue diverter receiver (140) and thus the tongue diverter (200), to separate the tongue diverter (200) and the bite block (100). The bite block may then be removed from the patient's mouth and set aside. Lastly, the tongue diverter (200), which had until this time remained in the patient's mouth, may be gently removed.

In another series of embodiments, an immobilization bite block system (10) system may include a bite block (100) having an integral bite-block airway passage (110), a dental interface (130) that is substantially ring-shaped and oriented parallel to a direction of bite. Such a dental interface (130) may have a maxillary and a mandibular dental engagement, releasably and directly engageable with a plurality of the maxillary and mandibular teeth of a patient, and a bite-block retention mask interface (120) releasably engageable with a retention mask (400).

In some embodiments, there may be a tongue diverter receiver (140) on the bite block (100), releasably engageable with a tongue diverter retainer (220) on a tongue diverter (200), to releasably engage the tongue diverter (200) and the bite block (100). The tongue diverter (200) may exert a direct force on a tongue when the tongue diverter retainer (220) is in use in place in the bite block (100), in a direction selected from the group of directions consisting of: to the patient's left, to the patient's right, caudally, or a combination of these.

The retention mask (400) may again be thermoformable and have two generally parallel opposing surfaces, a first surface in direct contact with the skin of the patient during use, and a second surface in direct contact with an ambient atmosphere during use. A retainer means (300) cooperating with the bite block (100) and the second surface of the retention mask (400) may releasably secure the bite block (100) and the retention mask (400).

The direct force upon the tongue may be facilitated, in some embodiments, so as to exert a direct force on the tongue when the tongue diverter retainer (220) is in place in the bite block (100) during use, in a direction selected from the group of directions consisting of: to the patient's left, to the patient's right, caudally, or a combination of these, and additionally the tongue diverter (200) may have a lingual surface shape selected from the group of lingual surface shapes consisting of; curved to the patient's left relative to an imaginary vertical passing through the bite block (100), curved to the patient's right relative to the imaginary vertical passing through the bite block (100), and substantially orthogonal to the imaginary vertical passing through the bite block (100).

As mentioned earlier, the retainer means (300) may be releasably engageable with the bite block (100) and the retention means (400) by means of an interface retainer (320) releasably forming the first surface of the retention mask (400) to the bite block (100) by pinching the retention mask (400) against the bite block (100).

In yet another series of embodiments, a bite block immobilization system (10) may include a bite block (100) having an integral bite-block airway passage (110), a dental interface (130) substantially ring-shaped and oriented parallel to a direction of bite and having a maxillary and a mandibular dental engagement, releasably and directly engageable with a plurality of the maxillary and mandibular teeth of a patient, and a bite-block retention mask interface (120) releasably engageable with a retention mask (400).

There may also be a tongue diverter receiver (140) on the bite block (100) releasably engageable with a tongue diverter retainer (220) on a tongue diverter (200) to releasably engage the tongue diverter (200) and the bite block (100), and further, the tongue diverter (200) may have a lingual surface shape selected from the group of lingual surface shapes consisting of; curved to the left relative to an imaginary vertical midline passing through the bite block (100), curved to the right relative to the imaginary vertical midline passing through the bite block (100), and substantially orthogonal to the imaginary vertical midline passing through the bite block (100).

In such embodiments, the tongue diverter (200) may exert a direct force on a tongue when the tongue diverter retainer (220) is in place during use in the bite block (100), in a direction selected from the group of directions consisting of: only to the patient's left, only to the patient's right, only caudally, or a combination of these.

Again, as discussed earlier, the retention mask (400) may be thermoformable and have two generally parallel opposing surfaces, a first surface in contact with the skin of the patient during use, and a second surface in contact with an ambient atmosphere during use. The bite block (100) may contact the retention mask (400) only on the first surface, and does not extend partially to, to, or through the second surface.

There may be a retainer means (300), often configured as a manually releasable resilient clip, cooperating with the bite block (100) and the second surface of the retention mask (400) to releasably secure the bite block (100) and the retention mask (400), where the retainer means (300) molds the first and second surfaces of the retention mask (400) around a bite block-retention mask interface lateral lip (120) formed on a lateral and anterior aspect of the bite block (100), by pinching the retention mask (400) between the bite block (100) and the retention means (300), and where the retention means (300) contacts the retention mask (400) only on the second surface, and does not extend partially to, to, or through the first surface,

The bite block (100) maybe disengageable, after the bite block (100) and the retention mask (400) are assembled into a usable conformation, from the retention mask (400) without residual effect on the retention mask (400) structure, and accordingly, the retainer means (300) may be fully releasable from the system (10) during therapeutic medical interventions.

In view of the written disclosure and figures provided herein, one skilled in the art will readily envision a method of using the system (10) for immobilization purposes. The steps of such a method may include first, placing a bite block (100) between and directly engageable with the maxilla and mandible of a human patient. Next, a thermally moldable retention mask (400) having two generally parallel opposing surfaces, a first surface in contact with the skin of a patient during use, and a second surface in contact with an ambient atmosphere during use, in a moldable state, may be placed over at least a portion of the patient.

One fitting the system (10) may then secure the retention mask (400) to the bite-block (100) with a releasable and fully removable, engageable retainer means (300), while the retention mask (400) is in a functional position, such that the retainer means (300) pinches the retention mask (400) between the bite block (100) and the retainer means (300).

Next the conversion of the retention mask (400) to a fixed state, either by cooling, chemical curing or some combination of both would be accomplished; and lastly, one would disengage and fully remove the releasable and fully removable engageable retainer means (300) from the system (10).

That, when configured in the embodiments above, neither any part of the bite block (100) or the retention means (300) remains on the second, i.e., outside surface of the retention mask, confers valuable advantages. In radiation beam therapy, therapists frequently wish to make directionality and field markings on the outside surface of the retention mask (400), which could be in the form of direct markings or with the application of adhesive markers. The embodiments described above may present an uninterrupted smooth second, i.e., outside surface, of the retention mask (400), to facilitate placing such markers.

Numerous alterations, modifications, and variations of the preferred embodiments disclosed herein will be apparent to those skilled in the art and they are all anticipated and contemplated to be within the spirit and scope of the disclosed specification. For example, although specific embodiments have been described in detail, those with skill in the art will understand that the preceding embodiments and variations can be modified to incorporate various types of substitute and or additional or alternative materials, relative arrangement of elements, order of steps and additional steps, and dimensional configurations. Accordingly, even though only few variations of the method and products are described herein, it is to be understood that the practice of such additional modifications and variations and the equivalents thereof, are within the spirit and scope of the method and products as defined in the following claims. The corresponding structures, materials, acts, and equivalents of all means or step plus function elements in the claims below are intended to include any structure, material, or acts for performing the functions in combination with other claimed elements as specifically claimed.

We claim:

1. A bite block immobilization system (10), comprising:
A bite block (100) having an integral bite-block airway passage (110), a dental interface (130), substantially ring-shaped and oriented parallel to a direction of bite and having a maxillary and a mandibular dental engagement, releasably and directly engageable with a plurality of the maxillary and mandibular teeth of a patient, and a bite-block retention mask interface (120) releasably engageable with a retention mask (400),
the retention mask (400) being thermoformable and having two generally parallel opposing surfaces, a first surface in contact with the skin of the patient during use, and a second surface in contact with an ambient atmosphere during use,
the bite block (100) contacting the retention mask (400) on the first surface, without extending partially to, to, or through the second surface, and
a retainer means (300) cooperating with the bite block (100) and the second surface of the retention mask (400) to releasably secure the bite block (100) and the retention mask (400),
and the retainer means (300) contacting the retention mask (400) on the second surface, and does not extend partially to, to, or through to the first surface.

2. The device according to claim 1, wherein the dental interface (130) is integrally formed with the bite-block as an outside surface of the bite block (100).

3. The device according to claim 1, wherein the dental interface (130) is moldable and fully removable from the bite block (10). removable from the bite block (100).

4. The device according to claim 1, wherein the dental interface (130) contacts eight or fewer teeth as found in a normal human full dentition set.

5. The device according to claim 1, wherein the dental interface (130) contacts 12 or fewer teeth as found in a normal human full dentition set.

6. The device according to claim 1, wherein a tongue diverter receiver (140) on the bite block (100) is releasably engageable with a tongue diverter retainer (220) on a tongue diverter (200) to releasably engage the tongue diverter (200) and the bite block (100).

7. The device according to claim 1, wherein the tongue diverter (200) exerts a direct force on the tongue when the tongue diverter retainer (220) is in place in the bite block (100) during use, in a direction selected from the group of directions consisting of: to a patient's left relative to an imaginary vertical passing through the bite block (100), to a patient's right relative to the imaginary vertical passing through the bite block (100), caudally, or a combination of these.

8. The device according to claim 6, wherein the tongue diverter (200) has a lingual surface shape selected from the group of lingual surface shapes consisting of; curved to the patient's left relative to an imaginary vertical passing through the bite block (100), curved to the patient's right relative to the imaginary vertical passing through the bite block (100), and substantially orthogonal to the imaginary vertical passing through the bite block (100).

9. The device according to claim 8, wherein the tongue diverter (200) has at least two lingual surface shapes selected from the group of lingual surface shapes consisting of; curved to the left relative to the imaginary vertical passing through the bite block (100), curved to the right relative to the imaginary vertical passing through the bite block (100), and substantially orthogonal to the imaginary vertical passing through the bite block (100).

10. The device according to claim 6, wherein a tongue diverter removal tool (240) is releasably engageable with the tongue diverter receiver (140) to releasably disengage the tongue diverter (200) and the bite block (100) while the bite block (100) remains engaged with the maxillary or mandibular teeth, or both the maxillary and mandibular teeth.

11. The device according to claim 1, wherein the retainer means (300) is a manually releasable resilient clip.

12. The device according to claim 1, wherein the retainer means (300) is releasably engageable with the bite block (100) and the retention means (400) by means of an interface retainer (320) releasably securing the bite block (100) to the first surface of the retention mask (400) by pinching the retention mask (400) against the bite block (100).

13. The device according to claim 1, wherein the retainer means (300) is fully releasable from the system (10) during therapeutic medical interventions.

14. The device according to claim 1, wherein the retainer means (300) molds the first and second surfaces of the retention mask around a bite block-retention mask interface lateral lip (120) formed on a lateral and anterior aspect of the bite block (100).

15. The device according to claim 1, wherein the bite block (100) is disengageable, after the bite block (100) and the retention mask (400) are assembled into a usable conformation, from the retention mask (400), without residual effect on the retention mask (400) structure.

16. A bite block immobilization system (10), comprising:
A bite block (100) having an integral bite-block airway passage (110), a dental interface (130) substantially ring-shaped and oriented parallel to a direction of bite and having a maxillary and a mandibular dental engagement, releasably and directly engageable with a plurality of the maxillary and mandibular teeth of a patient, and a bite-block retention mask interface (120) releasably engageable with a retention mask (400),
a tongue diverter receiver (140) on the bite block (100) releasably engageable with a tongue diverter retainer (220) on a tongue diverter (200) to releasably engage the tongue diverter (200) and the bite block (100),
wherein the tongue diverter (200) exerts a direct force on a tongue when the tongue diverter retainer (220) is in place in use in the bite block (100), in a direction selected from the group of directions consisting of: to the patient's left, to the patient's right, caudally, or a combination of these,
the retention mask (400) being thermoformable and having two generally parallel opposing surfaces, a first surface in direct contact with the skin of the patient during use, and a second surface in direct contact with an ambient atmosphere during use,
and a retainer means (300) cooperating with the bite block (100) and the second surface of the retention mask (400) to releasably secure the bite block (100) and the retention mask (400).

17. The device according to claim 16, wherein the tongue diverter (200) exerts a direct force on the tongue when the tongue diverter retainer (220) is in place in the bite block (100) during use, in a direction selected from the group of directions consisting of: to the patient's left, to the patient's right, caudally, or a combination of these, and
wherein the tongue diverter (200) has a lingual surface shape selected from the group of lingual surface shapes consisting of; curved to the patient's left relative to an imaginary vertical passing through the bite block (100), curved to the patient's right relative to the imaginary vertical passing through the bite block (100), and substantially orthogonal to the imaginary vertical passing through the bite block (100).

18. The device according to claim 1, wherein the retainer means (300) is releasably engageable with the bite block (100) and the retention means (400) by means of an interface retainer (320) releasably forming the first surface of the retention mask (400) to the bite block (100) by pinching the retention mask (400) against the bite block (100).

19. A bite block immobilization system (10), comprising:
A bite block (100) having an integral bite-block airway passage (110), a dental interface (130) substantially ring-shaped and oriented parallel to a direction of bite and having a maxillary and a mandibular dental engagement, releasably and directly engageable with a plurality of the maxillary and mandibular teeth of a patient, and a bite-block retention mask interface (120) releasably engageable with a retention mask (400),
a tongue diverter receiver (140) on the bite block (100) releasably engageable with a tongue diverter retainer (220) on a tongue diverter (200) to releasably engage the tongue diverter (200) and the bite block (100),
the tongue diverter (200) having a lingual surface shape selected from the group of lingual surface shapes consisting of; curved to the left relative to an imaginary vertical midline passing through the bite block (100), curved to the right relative to the imaginary vertical midline passing through the bite block (100), and substantially orthogonal to the imaginary vertical midline passing through the bite block (100),
wherein the tongue diverter (200) exerts a direct force on a tongue when the tongue diverter retainer (220) is in place during use in the bite block (100), in a direction selected from the group of directions consisting of: only to the patient's left, only to the patient's right, only caudally, or a combination of these,
the retention mask (400) being thermoformable and having two generally parallel opposing surfaces, a first surface in contact with the skin of the patient during use, and a second surface in contact with an ambient atmosphere during use,
the bite block (100) contacts the retention mask (400) only on the first surface, and does not extend partially to, to, or through the second surface, and
a retainer means (300) cooperating with the bite block (100) and the second surface of the retention mask (400) to releasably secure the bite block (100) and the retention mask (400), wherein the retainer means (300) molds the first and second surfaces of the retention mask (400) around a bite block-retention mask interface lateral lip (120) formed on a lateral and anterior aspect of the bite block (100), by pinching the retention mask (400) between the bite block (100) and the retention means (300), wherein the retention means (300) contacts the retention mask (400) only on the second surface, and does not extend partially to, to, or through the first surface,
wherein the bite block (100) is disengageable, after the bite block (100) and the retention mask (400) are assembled into a usable conformation, from the retention mask (400) without residual effect on the retention mask (400) structure.
wherein the retainer means (300) is fully releasable from the system (10) during therapeutic medical interventions.

20. The device according to claim 1, wherein the bite block (100) reversibly snaps into a pocket formed in the first surface by the molding of the retention mask (400) around the bite block (100) by the retainer means (300).

21. A method for forming a bite block immobilization system (10) comprising the steps of:
1. placing a bite block (100) between and directly engageable with the maxilla and mandible of a human patient;
2. placing a thermally moldable retention mask (400) having two generally parallel opposing surfaces, a first surface in contact with the skin of a patient during use, and a second surface in contact with an ambient atmosphere during use, in a moldable state, over at least a portion of the patient;
3. securing the retention mask (400) to the bite-block (100) with a releasable and fully removable, engageable retainer means (300), while the retention mask (400) is in a functional position, such that the retainer means (300) pinches the retention mask (400) between the bite block (100) and the retainer means (300);
4. facilitating the conversion of the retention mask (400) to a fixed state; and
5. disengaging and fully removing the releasable and fully removable engageable retainer means (300).
